(19) United States
(12) Patent Application Publication
(10) Pub. No.: US 2004/0209900 A1
(43) Pub. Date: Oct. 21, 2004

(54) Treatment of lipodystrophy (lipid redistribution syndrome)

(76) Inventor: Stephen P. Hauptman, Elkins Park, PA (US)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(21) Appl. No.: 10/818,688

(22) Filed: Apr. 6, 2004

Related U.S. Application Data
(60) Provisional application No. 60/462,972, filed on Apr. 15, 2003.

(51) Int. Cl. A61K 31/522

(52) U.S. Cl. 514/263.38

(57) ABSTRACT

A pharmaceutical composition for treating lipodystrophy and a method of treating lipodystrophy with the pharmaceutical composition. The composition comprising a therapeutically effective amount, e.g., 400 mg. three times per day, of pentoxifylline and a pharmaceutically acceptable carrier.

### CROSS-REFERENCE TO RELATED APPLICATIONS

This application claims priority from provisional U.S. Patent Application No. 60/462,972, filed on Apr. 15, 2003.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### BACKGROUND OF THE INVENTION

1. Field of Invention

This invention relates generally to a method of treating lipodystrophy or fat redistribution syndrome through the use of pentoxifylline.

2. Description of Related Art

Persons taking steroids, e.g., prednisone, methyl prednisone, dexamethasone, or having diabetes, Cushing's disease or HIV frequently exhibit symptoms of lipodystrophy.

As is known by those skilled in the art lipodystrophy is perhaps the most common presentation of long-term HIV positive patients. It is also frequently found in diabetic patients and patients with Cushing's Syndrome (idiopathic or secondary to steroids). The syndrome consists of some or all of the following characteristics: loss of facial fat (fat wasting of face so patient looks older and wrinkled); fat loss of buttocks, arms and legs resulting in the loss of the gluteus, thinning of the arms and legs, and prominent leg veins resembling varicosities in young men; accumulation of fat, e.g., central obesity with marked increase in deep fat (ventral fat pad accumulation below the abdominal musculature) versus the more usual subcutaneous fat accumulation; and the so-called “Buffalo Hump” over the posterior neck (fat accumulation enlarging of the cervico-dorsal fat pad); and fat accumulation on anterior neck as well.

A need exists for the effective treatment of lipodystrophy or fat redistribution syndrome.

### BRIEF SUMMARY OF THE INVENTION

The subject invention is a method of treating lipodystrophy. The method comprises administering a therapeutically effective amount of pentoxifylline to a being with lipodystrophy.

### DETAILED DESCRIPTION OF THE INVENTION

In diabetic patients who are HIV positive with peripheral vascular disease and intermittent claudication (pain in calves with exertion and relief with rest, similar to chest angina), using pentoxifylline for claudication, many of the signs and symptoms of lipodystrophy disappeared or were ameliorated. The results looked anti-aging, e.g., facial lines decreased and/or disappeared, and central fat deposition resolved or remitted and leg vein prominence diminished or disappeared. It is believed that 80-85% of patients respond favorably with amelioration some or all of their symptoms.

In accordance with one aspect of this invention one can effectively treat lipodystrophy in a living being, e.g., a human being, by administering a therapeutically effective amount of pentoxifylline to the being, with the administration being accomplished via any effective modality. For example, in accordance with one exemplary aspect of this invention 400 mg. of pentoxifylline may be orally administered three times per day to effectively treat a person having lipodystrophy. It should be noted that in accordance with another aspect of this invention pentoxifylline may be administered for reducing the adverse cosmetic effect, e.g., skin tissue wrinkling effects, of aging or other conditions resulting in the inappropriate loss or deposition of fat, e.g., loss of facial fat or increase abdominal girth. The application of pentoxifylline may also be administered for reducing the abnormal accumulation of fat in the cervico-thoracic area or in the abdominal region. Further still, the application of pentoxifylline may also be administered for reducing the loss of fat in the arms, legs and buttock or other sites mentioned above of those persons having other syndromes exhibiting the same symptoms as lipodystrophy.

It is believed that pentoxifylline has its lipodystrophy treating and/or anti-aging and/or fat accumulation retarding and/or fat atrophy reduction effects because it lowers tumor necrosis factor (TNF), i.e., is a TNF inhibitor, a mediator of inflammation and possible fat atrophy and fat accumulation. High levels of tumor necrosis factor may result in lipodystrophy. There are other such TNF inhibitors on the market. However, they are expensive (e.g., $1,200 to $1,800 a month) and must be administered either subcutaneously or intravenously for Rheumatoid Arthritis and Inflammatory Bowel Disease. One or more of such TNF inhibitors, like pentoxifylline, may also have applicability for accomplishing the lipodystrophy treating and/or anti-aging and/or fat accumulation retarding effects, and as such are to be considered within the scope of this invention.

Without further elaboration the foregoing will so fully illustrate my invention that others may, by applying current or future knowledge, adopt the same for use under various conditions of service.

What is claimed is:

1. A method of treating lipodystrophy in a being comprising administering a therapeutically effective amount of pentoxifylline to the being.

2. The method of claim 1 wherein is administered orally.

3. The method of claim 1 wherein said therapeutically effective amount is 400 mg. three times per day.

4. A method of reducing facial wrinkles in a living being comprising administering a therapeutically effective amount of pentoxifylline to the being.

5. The method of claim 4 wherein is administered orally.

6. The method of claim 4 wherein said therapeutically effective amount is 400 mg. three times per day.

7. A method of reducing the abnormal accumulation of fat in either the cervico-thoracic area or in the abdominal region of a living being comprising administering a therapeutically effective amount of pentoxifylline to the being.

8. The method of claim 7 wherein is administered orally.

9. The method of claim 7 wherein said therapeutically effective amount is 400 mg. three times per day.

10. A method of reducing the abnormal loss of fat in the face, arms or in the legs of a living being comprising administering a therapeutically effective amount of pentoxifylline to the being.

11. The method of claim 10 wherein is administered orally.

12. The method of claim 10 wherein said therapeutically effective amount is 400 mg. three times per day.

13. A pharmaceutical composition for treating lipodystrophy comprising a therapeutically effective amount of pentoxifylline and a pharmaceutically acceptable carrier.

14. A pharmaceutical composition for reducing facial wrinkles comprising a therapeutically effective amount of pentoxifylline and a pharmaceutically acceptable carrier.

15. A pharmaceutical composition for reducing the abnormal accumulation of fat in either the cervico-thoracic area or in the abdominal region of a living being comprising a therapeutically effective amount of pentoxifylline and a pharmaceutically acceptable carrier.

16. A pharmaceutical composition for reducing the abnormal loss of fat in the arms or in the legs of a living being comprising a therapeutically effective amount of pentoxifylline and a pharmaceutically acceptable carrier.
